(19) United States
(12) Patent Application Publication
(10) Pub. No.: US 2002/0179099 A1
(43) Pub. Date: Dec. 5, 2002

(54) Hypodermic needle recapping device for surgical procedures

(76) Inventor: Denise Paxton, Virginia Beach, VA (US)

Correspondence Address: Denise Paxton, 212 76 th Street, Virginia Beach, VA, 23451, US

(21) Appl. No.: 09/841,355

(22) Filed: Apr. 24, 2001

(51) Int. Cl. A61M 5/32

(52) U.S. Cl. 128/919; 604/192

(57) ABSTRACT

A device for safely recapping a hypodermic needle (24) on a syringe (22) using a one-handed operation during surgical procedures. The device includes a stainless steel housing having a tapered open proximal end and a square based open distal end. The encased, hollow shaft (20) houses the plastic elongated needle cap (26) in which the user inserts the exposed said hypodermic needle and syringe for the purpose of recapping by applying a downward pressure.

### CROSS-REFERENCE TO RELATED APPLICATIONS

Not applicable

### BACKGROUND

1. Field of Invention

This invention relates to hypodermic needle recapping devices used on the sterile field during surgical procedures using the one-handed method.

2. Description of Prior Art

In the surgical field it is against hospital policy and OSHA regulations to recap hypodermic needles using the two-handed method which resulted in needle sticks, exposing the user to such deadly diseases as HIV, AIDS and hepatitis. In order to prevent exposure to these diseases and loss of life, inventors created several types of needle recapping devices. U.S. Pat. No. 5,209,738 to Bruno (1993) discloses a device containing materials that cannot be sterilized by steam or chemicals. The adhesive and suction devices will not adhere safely on the surgical field and the base is unsteady. It is not reusable. U.S. Pat. No. 4,717,386 to Simmons (1988) discloses a one and two-handed method of recapping. The one-handed method shows a sheath reception which is not long enough to safely house the sheath of the needle. Adhesive materials make steam and chemical sterilization impossible.

U.S. Pat. No. 4,737,149 to Gililan (1988) can not be sterilized by chemicals or steam because of the adhesives. The sheath reception is not long enough to safely house the needle sheath. The base on another model is not steady and requires a two-handed method. U.S. patent D370,062 to Tucker (1996) has limited sterilization means.

All needle recapping devices heretofore developed suffer from the disadvantage of not being able to be sterilized by all three methods of sterilization, being gas, steam and chemicals.

### SUMMARY

In accordance with the present invention an instrument which safely recaps a hypodermic needle on a syringe by inserting the cap down a shaft housed on the surgical field using the one-handed method.

### OBJECTS AND ADVANTAGES

Accordingly, in addition to the objects, the advantages of the sterilization process described in the invention above are:

a) to provide an instrument which can be sterilized by gas, steam and chemicals;

b) to provide a durable, reusable instrument that can be included on every instrument tray used in surgery;

c) to provide a lifetime instrument with no repairs;

d) to provide an instrument which uses the one-handed method of recapping;

e) to provide an instrument which has a sturdy, weighted base for safe mounting on the surgical field;

f) to provide an instrument that safely houses the needle sheath.

Further objects and advantages will become apparent from a consideration of the ensuing description and drawings.

### DRAWING FIGURES

In the drawings, closely related figures have the same number.

FIG. 1 shows the left, right, front and back view which includes the shaft.

FIG. 2 shows the top view.

FIG. 3 shows the bottom view.

FIG. 4 shows a view when the needle has been recapped.

FIG. 5 shows the method of the recapping procedure.

### REFERENCE NUMERALS IN DRAWING

| | |
|---|---|
| 10 base | 12 side |
| 14 sloped neck | 16 top |
| 18 hole | 20 shaft |
| 22 syringe | 24 hypodermic needle |
| 26 needle cap | |

### DESCRIPTION—FIGS. 14

An isometric view of the present invention is illustrated in FIG. 1. The square shaped base 10 sits on a flat surface and is 40 mm. The four sides 12 which come up from the base 10 measure 35 mm and form the sloped neck 14 which is 25 mm. The sloped neck 14 joins at the top 16 which measures 11 mm. The hole 18 is 7 mm in diameter. The shaft 20 measures 57 mm longitudinally from the top 16 to the base 10. There is a 22 mm square open space at the base 10 which measures 11 mm high.

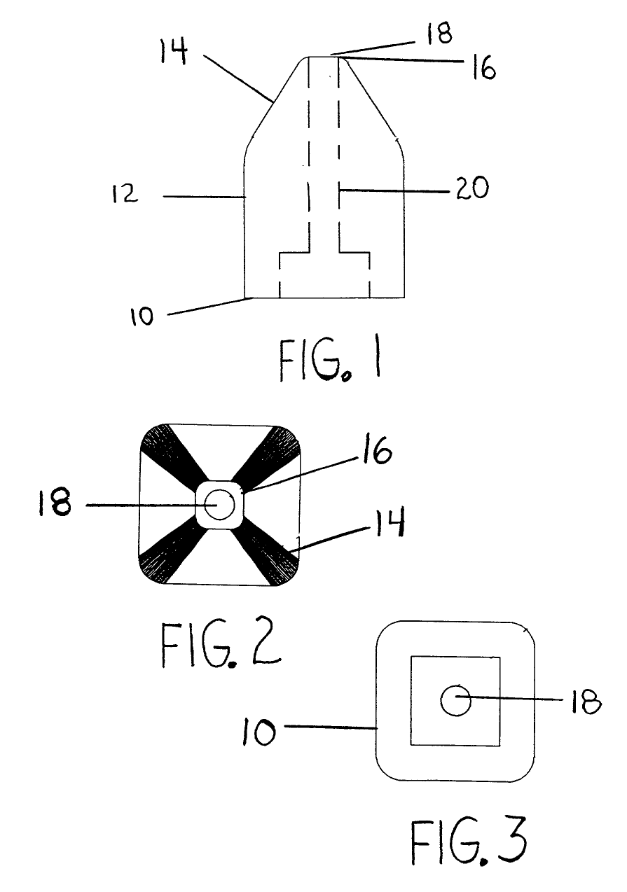

FIG. 2 shows a top view of the present invention. The hole 18 rests squarely at the top 16 which is joined by the four sloped necks 14.

FIG. 3 shows a bottom view. The base 10 has a width of 9 mm. The hole 18 can be seen from the bottom of the base 10.

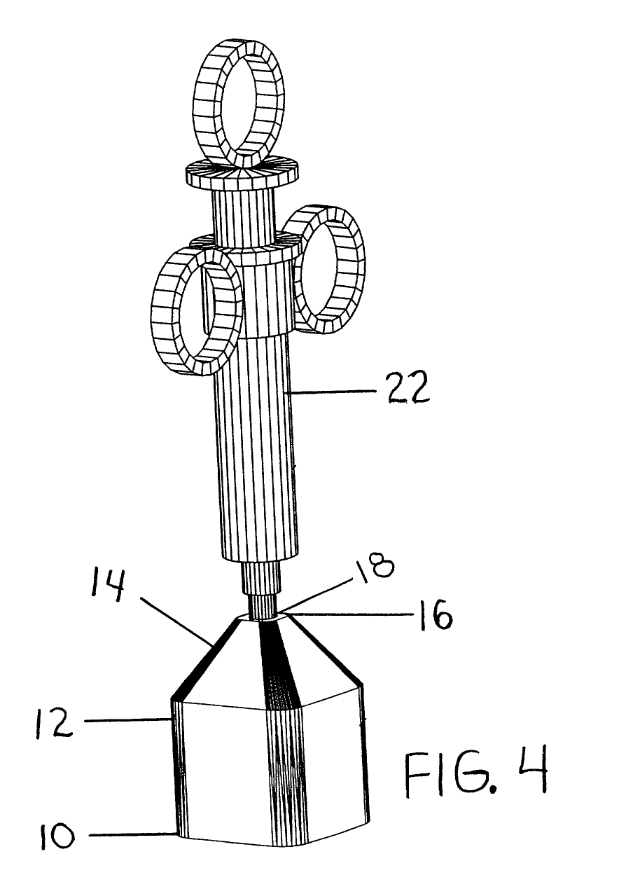

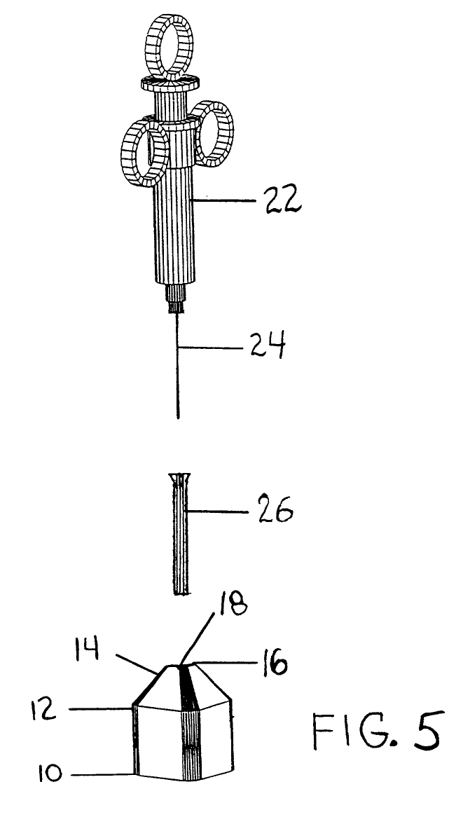

FIG. 4 shows the syringe 22 properly inserted in the hole 18 at the top 16 of the instrument.

FIG. 5 shows the method of inserting the syringe 22 with the attached needle 24 and cap 26 lined up to the opening of the hole 18 at the top 16 of the instrument.

Advantages

From the description above, a number of advantages of my needle recapping device become evident:

(a) The sloped neck makes insertion of the cap and needle easy.

(b) The sturdy base and weight assures it will not topple and will rest securely on any surface.

(c) The stainless steel material is reusable and can be sterilized by gas, steam or chemicals. It can be included on every surgical instrument tray to provide safe recapping for the surgical technician during surgery.

(d) The simple design make for easy, cheap and fast production.

(e) The durability of the stainless steel material make it of lifetime use with no repairs.

(f) At 600,000 needle sticks a year in the U.S. it saves hospitals costly and timely follow up visits for testing.

(g) It will save insurance companies money and timely claims.

(h) It will save hospitals from costly OSHA fines for improper recapping.

(l) Not only can it be used in surgery but in doctor's offices, veterinarian offices and hospital rooms.

(j) It can be used with all pre-existing needles in service at hospitals.

(k) It can save a life by preventing a needle stick.

Operation—FIG. 4,5

As seen in FIG. 5, the base 10 sits on a flat surface on the surgical field. After the hypodermic needle 24 has been used, the needle cap 26 is lined up to the opening of the hole 18. The cap 26 is inserted down the shaft 20 and is caught by the opening of the hole 18 which is smaller than the needle cap head. The syringe 22 and hypodermic needle 24 are lined up to the cap 26 which is resting vertically in the shaft 20. The syringe 22 and attached needle 24 are pushed in a downward motion into the cap 26. When this downward pressure is applied, the needle 24 is recapped. The syringe 22 and recapped needle 24 are lifted up and placed on a flat surface of the surgical field or be stored in an upright position in the recapping device.

FIG. 4 shows the syringe 22 after being recapped, resting vertically in the needle recapping device.

Conclusion, Ramifications, and Scope

Accordingly, the reader will see that the needle recapping device can be used to safely recap a needle using the one-handed method. It can be sterilized by any three methods of sterilization, being steam, gas and chemicals. It is reusable and can be included on every surgical instrument tray. Furthermore, the recapping device has the additional advantages in that

It provides an inexpensive, easy and fast production of the model because of the simple design;

It provides a lifetime service with no repairs because of the stainless steel material;

Its applications are for sterile use in doctor's offices, hospital rooms and veterinarian's offices.

Although the description above contains many specifications, these should not be construed as limiting the scope of the invention but merely providing illustrations of some of the presently preferred embodiments of this invention. For example, the base could have other shapes, such as circular.

Thus, the scope of the invention should be determined by the appended claims and their legal equivalents, rather than the examples given.

I claim:

1. A device which enables the user to safely recap a hypodermic needle on a syringe using a one-handed operation during surgical procedures, comprising:
A stainless steel housing having a tapered open proximal end and a square based open distal end;
An encased hollow shaft which houses the plastic elongated needle cap in which the user inserts exposed said needle and syringe for the purpose of recapping by applying a downward pressure.
